(19) United States
(12) Patent Application Publication
(10) Pub. No.: US 2020/0008692 A1
(43) Pub. Date: Jan. 9, 2020

(54) BLOOD PRESSURE DATA PROCESSING APPARATUS, BLOOD PRESSURE DATA PROCESSING METHOD, AND PROGRAM

(71) Applicant: OMRON HEALTHCARE Co., Ltd., Muko-shi (JP)

(72) Inventors: Keiichi OBAYASHI, Kyoto (JP); Hiroshi NAKAJIMA, Kyoto (JP); Naoki TSUCHIYA, Kyoto (JP); Hirotaka WADA, Kyoto (JP)

(73) Assignee: OMRON HEALTHCARE Co., Ltd., Muko-shi (JP)

(21) Appl. No.: 16/572,211

(22) Filed: Sep. 16, 2019

Related U.S. Application Data
(63) Continuation of application No. PCT/JP2018/009581, filed on Mar. 12, 2018.

(30) Foreign Application Priority Data
Mar. 14, 2017 (JP) 2017-048966

(51) Int. Cl. A61B 5/021; A61B 5/00

(52) U.S. Cl. CPC A61B 5/02125; A61B 5/7264; A61B 5/742; A61B 5/02116

(57) ABSTRACT

A blood pressure data processing apparatus includes: a blood pressure data acquisition unit to acquire blood pressure data; a blood pressure surge candidate detection unit detecting a blood pressure waveform that is a blood pressure surge candidate based on the blood pressure data; a blood pressure waveform extraction unit extracting a blood pressure waveform of one or more heartbeats; a waveform feature amount calculation unit calculating a waveform feature amount for each blood pressure waveform of one heartbeat isolated from the blood pressure waveform of one or more heartbeats, or for an average blood pressure waveform obtained by averaging the blood pressure waveforms of one heartbeat isolated from the blood pressure waveform of one or more heartbeats; and a blood pressure surge identification unit identifying whether or not the blood pressure waveform that is the blood pressure surge candidate is a blood pressure surge based on the waveform feature amount.

### TECHNICAL FIELD

The present invention relates to a technique for processing blood pressure data.

### BACKGROUND ART

It is known that in a patient affected by sleep apnea syndrome (SAS), when breathing is resumed after apnea, the blood pressure suddenly rises and thereafter falls. Hereinafter, this sudden blood pressure fluctuation will be called a “blood pressure surge”. It is thought that indices relating to blood pressure surges that occur in a patient (e.g., number of instances in which blood pressure surges occur per unit time) are useful in diagnosing and treating a disease that increases the risk of onset of brain disease or vascular disease, such as SAS or high blood pressure.

In order to observe a blood pressure surge, a blood pressure measurement apparatus that can continuously measure blood pressure, such as a blood pressure measurement apparatus that can measure the blood pressure for each heartbeat, for example, is needed. The amount of blood pressure data obtained through continuous blood pressure measurement is large, and thus it is difficult for an expert such as a doctor or researcher to analyze the blood pressure data to extract the blood pressure surge. For this reason, development of a technique for automatically extracting a blood pressure surge from blood pressure data has been progressing. JP 2014-158956A discloses a method for detecting vascular symptoms of a patient using artery pressure waveform data. With this method, merely the presence or absence of vascular symptoms is evaluated, and thus it is not possible to find a blood pressure abnormality that occurs repeatedly, such as a blood pressure surge.

There is not sufficient knowledge about blood pressure surges, and thus it is difficult to judge whether a sudden blood pressure fluctuation included in the blood pressure data obtained through continuous blood pressure measurement is a blood pressure surge or noise.

### SUMMARY OF INVENTION

The present invention was made in view of the foregoing circumstances, and it is an object thereof to provide a blood pressure data processing apparatus, a blood pressure data processing method, and a program, according to which it is possible to judge whether or not a sudden blood pressure fluctuation included in the blood pressure data obtained through continuous blood pressure measurement is a blood pressure surge.

In a first aspect of the present invention, a blood pressure data processing apparatus includes: a blood pressure data acquisition unit configured to acquire blood pressure data; a blood pressure surge candidate detection unit configured to detect a blood pressure waveform that is a blood pressure surge candidate based on the blood pressure data; a blood pressure waveform extraction unit configured to extract a blood pressure waveform of one or more heartbeats from the blood pressure waveform that is the blood pressure surge candidate; a waveform feature amount calculation unit configured to calculate a waveform feature amount for each blood pressure waveform of one heartbeat isolated from the blood pressure waveform of one or more heartbeats, or for an average blood pressure waveform obtained by averaging the blood pressure waveforms of one heartbeat isolated from the blood pressure waveform of one or more heartbeats; and a blood pressure surge identification unit configured to identify whether or not the blood pressure waveform that is the blood pressure surge candidate is a blood pressure surge based on the waveform feature amount.

In a second aspect of the present invention, the waveform feature amount includes a plurality of types of waveform feature amounts, and the blood pressure surge identification unit identifies whether or not the blood pressure waveform that is the blood pressure surge candidate is a blood pressure surge based on the plurality of types of waveform feature amounts and a boundary set in a feature space.

In a third aspect of the present invention, the blood pressure waveform of the blood pressure surge candidate includes a rising portion and a falling portion that is subsequent to the rising portion, and the blood pressure waveform extraction unit extracts the blood pressure waveform of one or more heartbeats from the rising portion of the blood pressure waveform that is the blood pressure surge candidate.

In a fourth aspect of the present invention, the waveform feature amount is based on at least one of a time interval from a time of a diastolic peak to a time of a systolic peak, a time interval from the time of the diastolic peak to a time of a dicrotic peak, a time width of the systolic peak, a total pulse time, an amplitude of the systolic peak, and an amplitude of the dicrotic peak.

In a fifth aspect of the present invention, the waveform feature amount includes a waveform feature amount obtained based on a ratio between the time width of the systolic peak and the total pulse time.

In a sixth aspect of the present invention, the waveform feature amount calculation unit performs pre-processing including a first derivative or a second derivative on the blood pressure waveform of one or more heartbeats, and specifies the diastolic peak, the systolic peak, and the dicrotic peak based on the waveform obtained through the pre-processing.

In a seventh aspect of the present invention, the blood pressure data processing apparatus further includes: a display unit configured to display a blood pressure waveform identified by the blood pressure surge identification unit as not being a blood pressure surge; and a reception unit configured to receive an instruction indicating whether or not the displayed blood pressure waveform is a blood pressure surge.

In an eighth aspect of the present invention, the blood pressure data processing apparatus further includes: a clustering unit configured to generate a class by performing clustering on blood pressure waveforms of one or more heartbeats included in blood pressure waveform identified by the blood pressure surge identification unit as not being a blood pressure surge; and an output unit configured to output information including a blood pressure waveform of one or more heartbeats representing the class.

According to the first aspect, it is determined whether or not the blood pressure waveform that is a blood pressure surge candidate is a blood pressure surge based on the waveform feature amount for each blood pressure waveform of one heartbeat included in the blood pressure waveform that is the blood pressure surge candidate, or for the average blood pressure waveform. Accordingly, it is possible to determine whether or not the blood pressure waveform that is the blood pressure surge candidate is a blood pressure surge without any human intervention.

According to the second aspect, a boundary for identifying a blood pressure surge is set in the feature space in advance. Accordingly, it is possible to determine whether or not a blood pressure waveform is a blood pressure surge, with a small amount of processing.

According to the third aspect, a waveform feature amount is calculated for each blood pressure waveform of one heartbeat included in a rising portion of a blood pressure surge, or for an average blood pressure waveform. Accordingly, a causal factor of the blood pressure surge can be accurately identified.

According to the fourth aspect, a waveform feature amount obtained based on at least one of the time interval from the time of the diastolic peak to the time of the systolic peak, the time interval from the time of the diastolic peak to the dicrotic peak, the time width of the systolic peak, total pulse time, the amplitude of the systolic peak, and the amplitude of the diastolic peak is used. Accordingly, a causal factor that caused the blood pressure surge can be accurately identified.

According to the fifth aspect, the waveform feature amount obtained based on the ratio between the time span of the systolic peak and the total pulse time is used. Accordingly, a causal factor that caused the blood pressure surge can be accurately identified.

According to the sixth aspect, pre-processing including a first derivative or a second derivative is performed on a blood pressure waveform of one heartbeat or more. Accordingly, it is easier to perform processing for specifying a feature point such as a diastolic peak, a systolic peak, and a dicrotic peak.

According to the seventh aspect, a blood pressure waveform identified by the blood pressure surge identification unit as not being a blood pressure surge is displayed. Accordingly, an expert can determine whether or not the displayed blood pressure waveform is a blood pressure surge.

According to the eighth aspect, clustering is performed on blood pressure waveforms of one or more heartbeats included in the blood pressure waveform determined by the blood pressure surge determination unit as not being a blood pressure surge, and information including a blood pressure waveform representing the class obtained as a result of clustering is output. Accordingly, a new class can be presented to the expert, and it is possible to discover a causal factor other than causal factors that have already been defined.

That is, according to the present invention, it is possible to provide a blood pressure data processing apparatus, a blood pressure data processing method, and a program, according to which it is possible to judge whether or not a sudden blood pressure fluctuation included in blood pressure data obtained through continuous blood pressure measurement is a blood pressure surge.

### BRIEF DESCRIPTION OF DRAWINGS

FIG. 1 is a block diagram showing a blood pressure data processing apparatus according to a first embodiment.

FIG. 2 is a block diagram showing an example of the blood pressure measurement apparatus shown in FIG. 1.

FIG. 3 is a side view showing an exterior of the blood pressure measurement unit shown in FIG. 2.

FIG. 4 is a cross-sectional view showing the blood pressure measurement unit shown in FIG. 2.

FIG. 5 is a cross-sectional view showing the blood pressure measurement unit shown in FIG. 2.

FIG. 6 is a diagram showing an example of a waveform of a blood pressure surge.

FIG. 7 is a block diagram showing a blood pressure surge identification unit shown in FIG. 1.

FIG. 8 is a diagram for illustrating a waveform feature amount.

FIG. 9 is a diagram for illustrating an example of a method for generating blood pressure surge identification data.

FIG. 10 is a diagram for illustrating an example of a method for generating blood pressure surge identification data.

FIG. 11 is a flowchart showing an example of processing of a blood pressure data processing apparatus according to a first embodiment.

FIG. 12 is a diagram showing a result of clustering.

FIG. 13 is a diagram showing a screen displaying a representative blood pressure waveform of a newly-generated class.

FIG. 14 is a diagram showing a feature space that has been labeled.

FIG. 15 is a block diagram showing an example of the hardware configuration of a blood pressure data processing apparatus of FIG. 1.

### DESCRIPTION OF EMBODIMENTS

Hereinafter, embodiments of the present invention will be described with reference to the drawings.

### First Embodiment

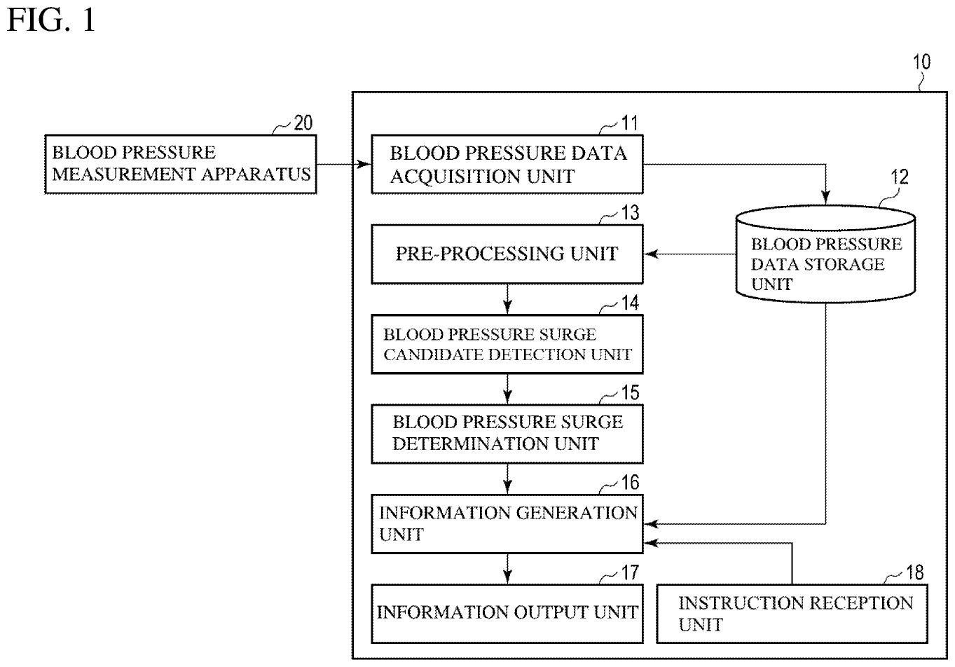

FIG. 1 schematically shows a blood pressure data processing apparatus 10 according to a first embodiment. As shown in FIG. 1, the blood pressure data processing apparatus 10 processes blood pressure data obtained in a blood pressure measurement apparatus 20 for measuring the blood pressure of a measurement subject (user). For example, the blood pressure data processing apparatus 10 can be equipped in a computer such as a personal computer or a server.

First, the blood pressure measurement apparatus 20 will be described. The blood pressure measurement apparatus 20 generates blood pressure data by continuously measuring the blood pressure of the measurement subject. Specifically, the blood pressure measurement apparatus 20 measures a pulse wave of an artery of the measurement subject and generates the blood pressure data by converting the measured pulse wave into blood pressure. The blood pressure data includes data on the blood pressure waveform corresponding to the waveform of the measured pulse wave. The blood pressure data may also further include chronological data of a blood pressure feature amount (blood pressure value). Although the blood pressure feature amounts include the systolic blood pressure (SBP) and the diastolic blood pressure (DBP) for example, there is no limitation to this. The maximum value of the pulse wave waveform of one heartbeat corresponds to the systolic blood pressure, and the minimum value of the pulse wave waveform of one heartbeat corresponds to the diastolic blood pressure.

In the first embodiment, the blood pressure measurement apparatus 20 measures the pressure pulse wave serving as the pulse wave through tonometry. Here, “tonometry” refers to a method in which a flat portion is formed in the artery by pressing the artery with an appropriate pressure from above the skin, and a pressure pulse wave is non-invasively measured using the pressure sensor in a state in which the interior and exterior of the artery are balanced. According to tonometry, it is possible to obtain a blood pressure value for each heartbeat.

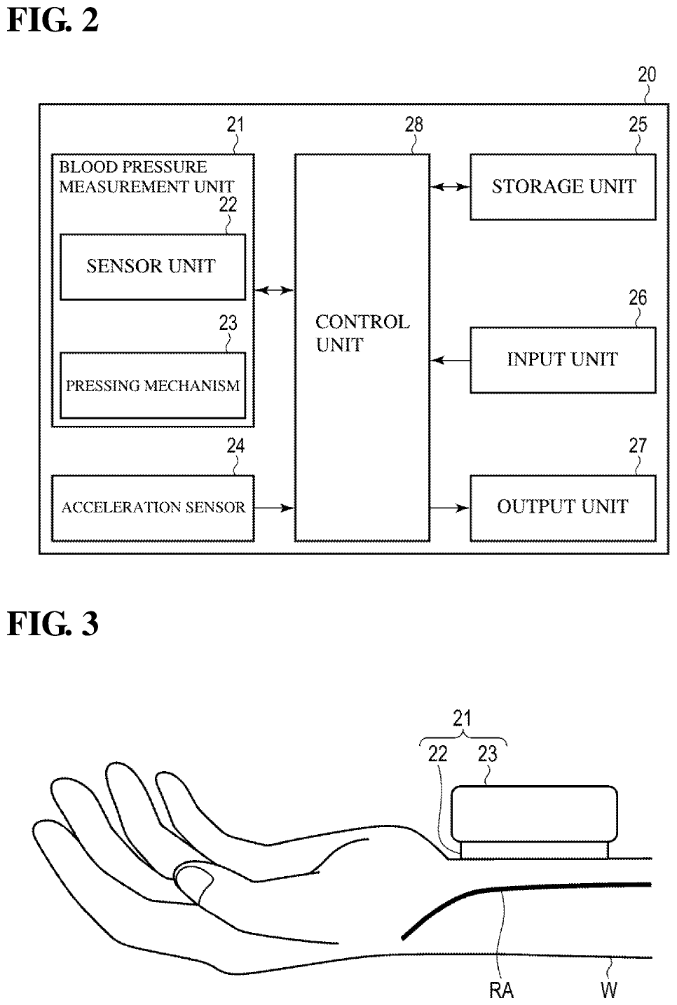

The blood pressure measurement apparatus 20 may also be a wearable apparatus to be attached to the measurement subject, or may be a stationary apparatus that performs blood pressure measurement in a state in which the upper arm of the measurement subject is placed on a fixed platform. In an example that will be described below with reference to FIGS. 2 to 5, the blood pressure measurement apparatus 20 is a wearable apparatus to be attached to a wrist of the measurement subject.

FIG. 2 schematically shows an example of the blood pressure measurement apparatus 20. The blood pressure measurement apparatus 20 shown in FIG. 2 includes a blood pressure measurement unit 21, an acceleration sensor 24, a storage unit 25, an input unit 26, an output unit 27, and a control unit 28. The control unit 28 controls the units of the blood pressure measurement apparatus 20. The function of the control unit 28 can be realized by a processor such as a CPU (central processing unit) executing a control program stored in a computer-readable storage medium such as a ROM (read-only memory).

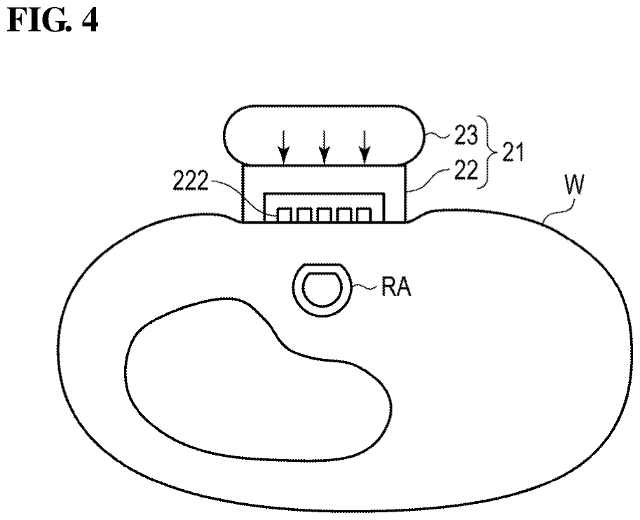

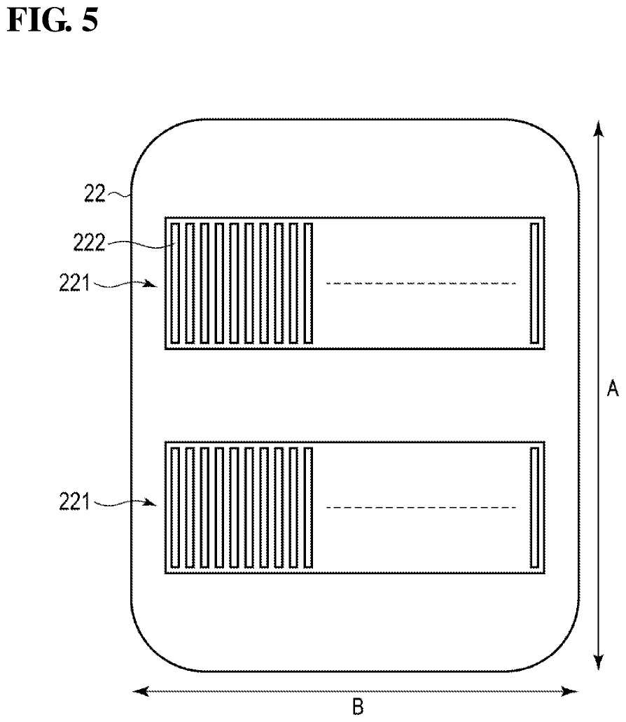

The blood pressure measurement unit 21 measures the pressure pulse wave of the radial artery. FIG. 3 is a side view showing a state in which the blood pressure measurement unit 21 is attached to a wrist W of the measurement subject using a belt (not shown), and FIG. 4 is a cross-sectional view schematically showing a structure of the blood pressure measurement unit 21. As shown in FIGS. 3 and 4, the blood pressure measurement unit 21 includes a sensor unit 22 and a pressing mechanism 23. The sensor unit 22 is arranged so as to come into contact with a part (in this example, the wrist W) in which the radial artery RA is present. The pressing mechanism 23 presses the sensor unit 22 to the wrist W. In tonometry, the pressure pulse wave and the blood pressure become equal under optimal pressing conditions.

FIG. 5 shows a surface on the side of the sensor unit 22 that comes into contact with the wrist W. As shown in FIG. 5, the sensor unit 22 includes one or more (in this example, two) pressure sensor arrays 221, and each pressure sensor array 221 has multiple (e.g., 46) pressure sensors 222 that are aligned in a direction B. The direction B is a direction that intersects a direction A in which the radial artery extends in a state in which the blood pressure measurement apparatus 20 is attached to the measurement subject. The arrangement of the pressure sensors 222 is not limited to the example shown in FIG. 5. Channel numbers serving as identification information have been added to the pressure sensors 222.

The pressure sensors 222 generate pressure data by measuring the pressure. Piezoelectric elements that convert pressure into electric signals can be used as the pressure sensors. The output signals of the piezoelectric elements are converted into digital signals at a predetermined (e.g., 125-Hz) sampling frequency, whereby the pressure data is obtained. The pressure pulse wave data corresponding to the above-described pulse wave data is generated based on the pressure data output from one pressure sensor (active channel) 222 selected adaptively from among the pressure sensors 222.

For example, the pressing mechanism 23 includes an air bag and a pump for adjusting the internal pressure of the air bag. When the pump is driven by the control unit 28 so as to increase the internal pressure of the air bag, the pressure sensor 222 is pressed to the wrist W due to the inflation of the air bag. Note that the pressing mechanism 23 is not limited to a structure using an air bag, and may also be realized by any structure in which the force pressing the pressuring sensor 222 to the wrist W can be adjusted.

The acceleration sensor 24 detects the acceleration acting on the blood pressure measurement apparatus 20 and generates acceleration data. For example, a triple-axial acceleration sensor can be used as the acceleration sensor 24. The detection of the acceleration is carried out in parallel with the blood pressure measurement.

The storage unit 25 includes a computer-readable storage medium. For example, the storage unit 25 includes a RAM (random access memory) and an auxiliary storage apparatus. The ROM stores the above-described control program. The RAM is used as a work memory by the CPU. The auxiliary storage apparatus stores various types of data including the blood pressure data generated by the blood pressure measurement unit 21, and the acceleration data generated by the acceleration sensor 24. The auxiliary storage apparatus includes a flash memory, for example. The auxiliary storage apparatus includes one or both of a storage medium built into the blood pressure measurement apparatus 20 and a removable medium such as a memory card.

The input unit 26 receives an instruction from the measurement subject. For example, the input unit 26 includes an operation button, a touch panel, and the like. The output portion 27 outputs information such as a pressure measurement result. For example, the output unit 27 includes a display apparatus such as a liquid crystal display apparatus.

According to the blood pressure measurement apparatus 20 having the above-described configuration, the blood pressure data and the acceleration data are obtained. For example, measurement is performed over an entire period during which the measurement subject is asleep (for example, one night), and the blood pressure data and acceleration data obtained through measurement are input to the blood pressure data processing apparatus 10.

Note that the blood pressure measurement apparatus 20 is not limited to a blood pressure measurement apparatus using tonometry, and may also be a blood pressure measurement apparatus of any type that can continuously measure blood pressure. For example, a blood pressure measurement apparatus that measures a volume pulse wave serving as a pulse wave may also be used. For example, the blood pressure measurement apparatus can measure a volume pulse wave of an artery using a photoelectric sensor or an ultrasonic probe, and can estimate the blood pressure based on the measured volume pulse wave. A blood pressure measurement apparatus may also be used which measures a pulse transit time (PTT), which is the transit time of a pulse wave being transmitted through an artery, and estimates the blood pressure based on the estimated pulse transit time.

Next, the blood pressure data processing apparatus 10 will be described. As shown in FIG. 1, the blood pressure data processing apparatus 10 includes: a blood pressure data acquisition unit 11, a blood pressure data storage unit 12, a pre-processing unit 13, a blood pressure surge candidate detection unit 14, a blood pressure surge determination unit 15, an information generation unit 16, an information output unit 17, and an instruction reception unit 18.

The blood pressure data acquisition unit 11 acquires the blood pressure data from the blood pressure measurement apparatus 20 and stores the acquired blood pressure data in the blood pressure data storage unit 12. The blood pressure data may also be provided from the blood pressure measurement apparatus 20 to the blood pressure data processing apparatus 10 through a removable medium such as a medium card. Alternatively, the blood pressure data may also be provided from the blood pressure measurement apparatus 20 to the blood pressure data processing apparatus 10 through communication (wired communication or wireless communication). Furthermore, the blood pressure data acquisition unit 11 may also further acquire acceleration data output from the acceleration sensor provided in the blood pressure measurement apparatus 20, or the like.

The pre-processing unit 13 receives the blood pressure data from the blood pressure data storage unit 12 and performs pre-processing on the blood pressure data. For example, the pre-processing unit 13 performs pre-processing such as smoothing, spike noise removal, and high-frequency component removal on the chronological data of the systolic blood pressure that is included in the blood pressure data or was generated based on the blood pressure data. The pre-processing may also include processing for detecting bodily movement of the measurement subject using the acceleration data and correcting the blood pressure data of a time segment in which bodily movement has been detected.

The blood pressure surge candidate detection unit 14 detects a blood pressure waveform that is a blood pressure surge candidate based on the pre-processed blood pressure data. For example, chronological data of the systolic blood pressure is used in processing for detecting the blood pressure waveform that is the blood pressure surge candidate. Any method may be used to detect a blood pressure waveform that is the blood pressure surge candidate. In the first embodiment, there is no limitation regarding what kind of blood pressure waveform is detected as the blood pressure surge candidate. The blood pressure surge candidates include a sudden blood pressure fluctuation that occurs due to a target causal factor (e.g., apnea), a sudden blood pressure fluctuation that occurs due to a causal factor other than a target causal factor, and a blood pressure fluctuation that is detected as a result of noise included in the blood pressure data. From among blood pressure surge candidates, a sudden blood pressure fluctuation caused by a target causal factor is designated as a blood pressure surge.

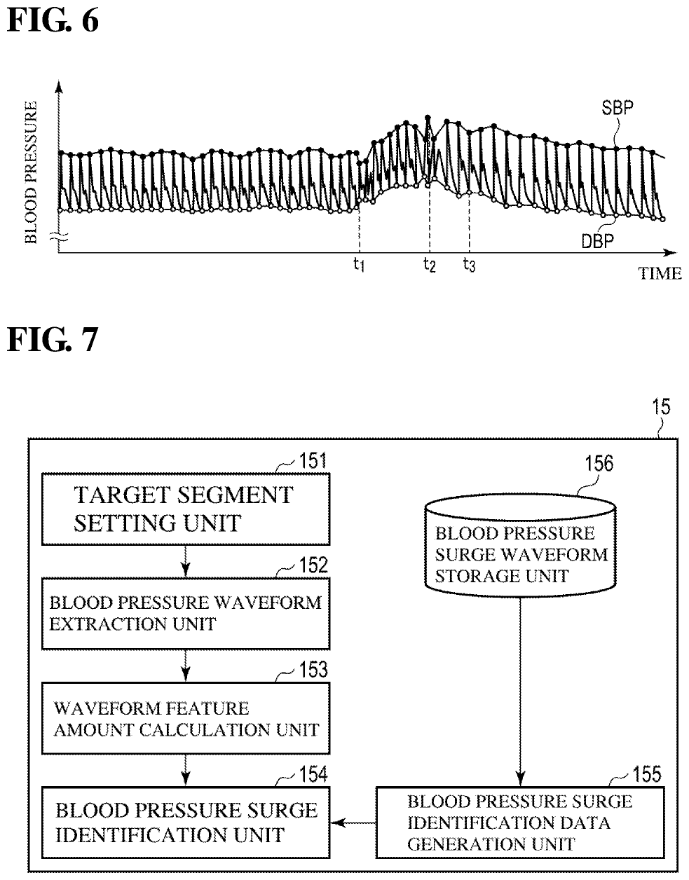

FIG. 6 shows an example of a blood pressure surge. In FIG. 6, the horizontal axis indicates time, and the vertical axis indicates blood pressure. The blood pressure waveform in the time segment (called a “surge segment”) from time t₁ to time t₃ corresponds to a blood pressure surge. In the surge segment, the blood pressure rises and thereafter falls. This kind of blood pressure fluctuation is detected as a blood pressure surge candidate. The blood pressure surge candidates can be managed using information including a time (called “peak time”) t₂ at which the blood pressure value reaches its maximum in the surge segment, a start time t₁ of the surge segment, and an end time t₃ of the surge segment. This information may also include the maximum blood pressure value in the surge segment.

The blood pressure surge determination unit 15 determines whether or not a blood pressure waveform that is a blood pressure surge candidate detected by the blood pressure surge candidate detection unit 14 is a blood pressure surge. The processing of the blood pressure surge determination unit 15 will be described in more detail later.

The information generation unit 16 generates the measurement blood pressure information. The information generation unit 16 can generate indices relating to blood pressure surges based on the blood pressure waveforms determined to be blood pressure surges by the blood pressure surge determination unit 15. For example, the indices relating to the blood pressure surges include: the number of instances of blood pressure surges per unit time, the average value of the maximum blood pressure values of the blood pressure surges, and the maximum value of the maximum blood pressure values of the blood pressure surges. Accordingly, it is possible to provide indices relating to blood pressure surges that occur in the measurement subject. Furthermore, the information generation unit 16 can generate various indices relating to the blood pressure, such as the average blood pressure value, based on the blood pressure data stored in the blood pressure data storage unit 12. Also, the information generation unit 16 may also generate a graph indicating the blood pressure waveform based on the blood pressure data, and add blood pressure surge position information (e.g., an arrow) indicating the position of the blood pressure surge on the graph.

The information output unit 17 outputs the measurement blood pressure information generated by the information generation unit 16. For example, the information output unit 17 generates image data including the measurement blood pressure information, and an image corresponding to the image data is displayed on the display apparatus. Also, the information output unit 17 generates the image data of the blood pressure waveform including the blood pressure surge position information, and an image corresponding to the image data is displayed on the display apparatus. Furthermore, the information output unit 17 may also output a blood pressure waveform determined by the blood pressure surge determination unit 15 as not being a blood pressure surge.

The instruction reception unit 18 receives an instruction from an operator (e.g., an expert). Examples of instructions include an instruction indicating whether or not a blood pressure waveform that has been determined by the blood pressure surge determination unit 15 as not being a blood pressure surge and has been displayed by the information output unit 17 is a blood pressure surge. For example, the expert observes the blood pressure waveform that has been determined by the blood pressure surge determination unit 15 as not being a blood pressure surge and has been displayed by the information output unit 17, determines whether or not the blood pressure waveform is a blood pressure surge, and inputs the determination result via the input apparatus. The information generation unit 16 may also re-calculate an index relating to the blood pressure surge based on the input determination result. By displaying a blood pressure waveform that has been determined by the blood pressure surge determination unit 15 as not being a blood pressure surge, it is possible for an expert to individually determine whether or not the blood pressure waveform is a blood pressure surge, and as a result, it is possible to provide more accurate measurement blood pressure information.

The blood pressure surge determination unit 15 will be described in detail.

FIG. 7 schematically shows an example of a configuration of the blood pressure surge determination unit 15. As shown in FIG. 7, the blood pressure surge determination unit 15 includes: a target segment setting unit 151, a blood pressure waveform extraction unit 152, a waveform feature amount calculation unit 153, a blood pressure surge identification unit 154, a blood pressure surge identification data generation unit 155, and a blood pressure surge waveform storage unit 156.

The target segment setting unit 151 sets a target segment for extracting a blood pressure waveform of one or more heartbeats from the blood pressure waveform that is the blood pressure surge candidate. For example, the rising time of the blood pressure waveform that is the blood pressure surge candidate is set as a target segment. The rising period of the blood pressure waveform that is the blood pressure surge candidate indicates the time segment from the start time t₁ to the peak time t₂. A portion of the rising segment may also be set as the target segment. Also, a portion or the entirety of the falling period may also be set as the target segment. The falling period indicates the time segment from the peak time t₂ to the end time t₃. The inventors of the present invention have confirmed that it is possible to accurately determine whether or not a blood pressure waveform that is a blood pressure surge candidate is a blood pressure surge by using the rising period of the blood pressure waveform that is the blood pressure surge candidate as the target segment. Accordingly, a portion or the entirety of the rising period of the blood pressure waveform that is the blood pressure surge candidate is preferably set as the target segment.

The blood pressure waveform extraction unit 152 extracts the blood pressure waveform of one or more heartbeats from the blood pressure waveform that is the blood pressure surge candidate in the target segment. The rising period of the blood pressure waveform that is the blood pressure surge candidate is typically about 5 to 25 seconds, and thus the blood pressure waveform over multiple heartbeats is extracted. Note that if the target segment is short, as in the case where a portion of the rising period of a blood pressure surge candidate is used as the target segment, a blood pressure waveform of less than two heartbeats is extracted in some cases as well.

The waveform feature amount calculation unit 153 extracts the waveform feature amount from the blood pressure waveform of one or more heartbeats calculated by the blood pressure waveform extraction unit 152. For example, the waveform feature amount calculation unit 153 isolates or extracts one or more blood pressure waveforms of one heartbeat from the blood pressure waveform of one or more heartbeats extracted by the blood pressure waveform extraction unit 152, and calculates the waveform feature amount for each isolated blood pressure waveform of one heartbeat. Also, the waveform feature amount calculation unit 153 may also generate the average blood pressure waveform obtained by averaging the isolated or extracted blood pressure waveforms of one heartbeat, and may calculate the waveform feature amount for the average blood pressure waveform. The waveform feature amount is calculated based on the shape of the blood pressure waveform of one heartbeat. The waveform feature amount includes one or more types of waveform feature amounts. In the first embodiment, multiple types of waveform feature amounts are used. The waveform feature amount can be expressed using a feature vector.

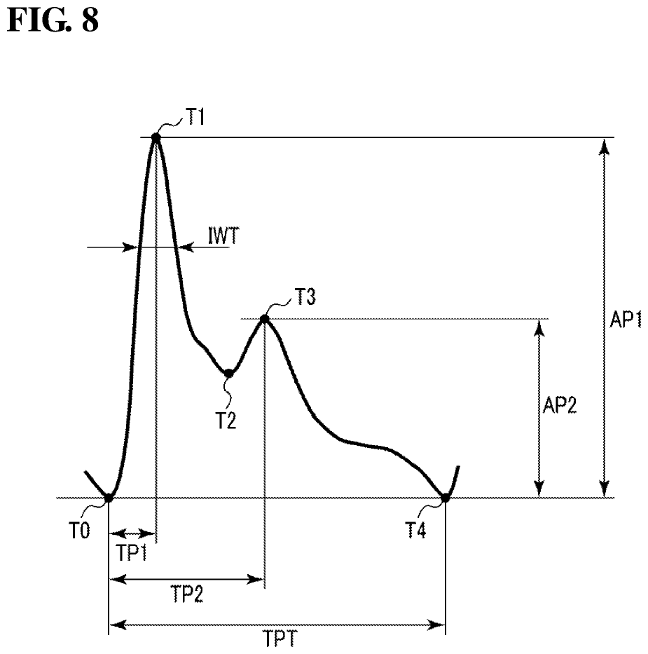

The waveform feature amount will be described with reference to FIG. 8. FIG. 8 illustrates a blood pressure waveform of one heartbeat. In FIG. 8, T0 is a point at which the blood pressure value (e.g., the value of the pressure pulse wave) reaches its minimum in the blood pressure waveform of one heartbeat. The point T0 is called the diastolic peak or the diastolic onset. T1 is the point at which the blood pressure value reaches its maximum in the blood pressure waveform of one heartbeat. The point T1 is called the systolic peak. T2 is an inflection point that appears after the point T1. Point T2 is called the dicrotic notch. T3 is an inflection point that appears after the point T2, that is, the point at which the blood pressure reaches a local maximum, and which appears after the maximum point T1. The point T3 is called the dicrotic peak. T4 is the point at which the blood pressure value reaches its minimum, and is a point that is the starting point of the blood pressure waveform of the next heartbeat. AP1 indicates the amplitude of the systolic peak, that is, a difference value obtained by subtracting the minimum value from the maximum value. AP2 indicates the amplitude of the dicrotic peak, that is, a difference value obtained by subtracting the minimum value from the second local maximum value. TP1 indicates the amount of time until the systolic peak, that is, the amount of time from the time of the minimum value to the time of the maximum value. Tp2 indicates the amount of time until the dicrotic peak, that is, the amount of time from the time of the minimum value to the time of the second local maximum value. TPT indicates the total pulse time, that is, the duration of the blood pressure waveform of one heartbeat. IWT indicates the time width of the systolic peak. For example, IWT is the inter-wave time, which has a value corresponding to two-thirds of the height (AP1) of the systolic peak. The waveform feature amount can be based on at least one of the parameters AP1, AP2, TP1, TP2, TPT, and IWT. For example, it is possible to use a waveform feature amount obtained based on TP1, IWT/TPT, TP1/TPT, TP2/TPT, (TP2-TP1)/TPT, AP2/AP1, or the like. In one example, two types of waveform feature amounts, namely IWT/TPT and AP2/AP1, are used. Note that the waveform feature amount may also be based on a parameter different from the above-described parameter.

The waveform feature amount calculation unit 153 may also perform pre-processing including a first derivative and/or a second derivative on the blood pressure waveform in order to specify feature points such as the points T0, T1, T2, T3, and T4. Processing for specifying the feature points is simplified by using the first derivative and/or the second derivative of the blood pressure waveform.

The blood pressure surge identification unit 154 identifies whether or not the blood pressure waveform that is the blood pressure surge candidate is a blood pressure surge based on the waveform feature amount calculated by the waveform feature amount calculation unit 153. The blood pressure surge identification unit 154 uses the blood pressure surge identification data generated by the blood pressure surge identification data generation unit 155 to perform identification. The blood pressure surge identification data will be described before giving a specific description of the blood pressure surge identification unit 154.

The blood pressure surge waveform storage unit 156 stores data of a typical blood pressure surge waveform. A blood pressure surge waveform in this context refers to the blood pressure waveform of one heartbeat, as shown in FIG. 8. The typical blood pressure surge waveform can be acquired due to an expert such as a doctor or a researcher analyzing the blood pressure data obtained for any measurement subject. For example, the typical blood pressure surge waveform is extracted from a blood pressure surge that is considered to be related to some kind of disease, as with a blood pressure surge that occurs when breathing is resumed after apnea.

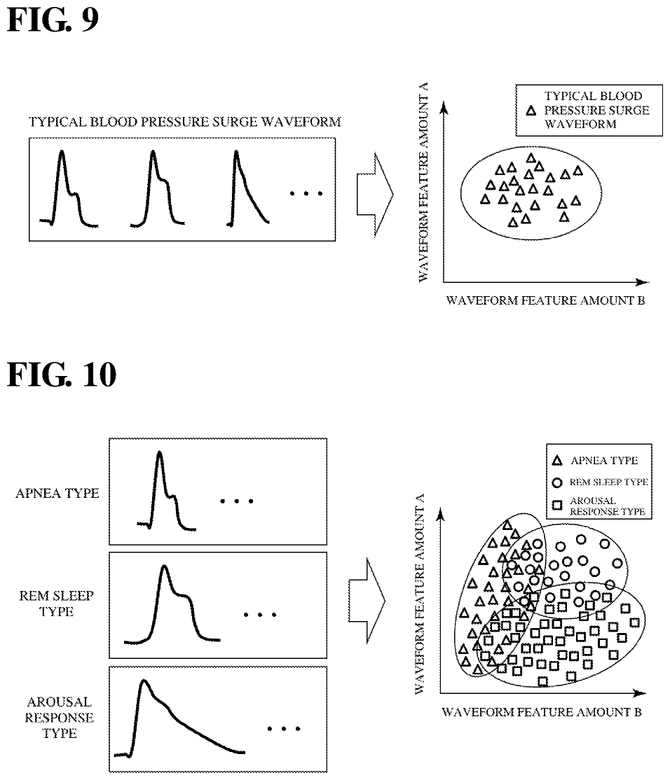

The blood pressure surge identification data generation unit 155 generates data (blood pressure surge identification data) to be used by the blood pressure surge identification unit 154 to perform identification, based on the blood pressure surge waveform data stored in the blood pressure surge waveform storage unit 156. The blood pressure surge identification data generation unit 155 calculates the waveform feature amount for each blood pressure surge waveform. Calculation of the waveform feature amount can be performed using a method similar to that described regarding the waveform feature amount calculation unit 153. The blood pressure surge identification data generation unit 155 sets a boundary line or a plane for identifying whether or not a blood pressure surge candidate is a blood pressure surge in a feature space based on the calculated waveform feature amounts. The blood pressure surge identification data generation unit 155 determines the boundary lines or boundary planes in which approximately 95.4% or approximately 99.7% of the data is included, as with a 2σ method or a 3σ method, in the feature space. The boundaries can be determined using, for example, the Mahalanobis distance, one-class support vector machine (SVM), or the like. If two types of blood pressure characteristic amounts are used, boundary lines such as those shown in FIG. 9 are set. The blood pressure surge identification data includes data indicating a boundary in a feature space.

In the above-described example, one data set of typical blood pressure surge waveforms is used. However, multiple data sets of typical blood pressure surge waveforms may also be used. Mainly apnea, REM (rapid eye movement) sleep, and an arousal response are thought of as causal factors that cause blood pressure surges during sleep. It is possible to judge which causal factor caused the blood pressure surge by measuring the sleep state and the blood pressure through PSG (polysomnography). A blood pressure surge occurs due to complex causal factors in some cases as well. For example, blood pressure surges occur due to apnea and REM sleep in some cases as well. Also, a blood pressure surge occurs due to apnea, REM sleep, and an arousal response in some cases as well. Note that there are also blood pressure surges for which causal factors cannot be specified. In the example shown in FIG. 10, three data sets of typical blood pressure surge waveforms are prepared, which correspond to three causal factors (classes), namely apnea, REM, and an arousal response. For each of the three classes, a boundary is set in the feature space using a method similar to that described above. Each of the boundaries can partially overlap with another boundary. In this example, the blood pressure surge identification data includes data indicating boundaries set in the feature space for each of the three classes.

The surge blood pressure identification unit 154 identifies whether or not the blood pressure waveform that is the blood pressure surge candidate is a blood pressure surge based on the position of the waveform feature amount calculated by the waveform feature amount calculation unit 153 in the feature space, and the boundary or plane set in the feature space. Specifically, the blood pressure surge identification unit 154 identifies whether or not the blood pressure waveform that is the blood pressure surge candidate is a blood pressure surge based on whether the feature vector is inside or outside of the boundary in the feature space. If multiple blood pressure waveforms of one heartbeat have been extracted by the blood pressure waveform extraction unit 152, the blood pressure surge identification unit 154 performs identification through majority decision, for example. Specifically, if the number of feature vectors located inside of the boundary in the feature space is greater than the number of feature vectors located outside of the boundary in the feature space, the blood pressure surge identification unit 154 identifies that the blood pressure waveform that is the blood pressure surge candidate is a blood pressure surge, and if the number of feature vectors located inside of the boundary in the feature space is less than the number of feature vectors located outside of the boundary in the feature space, the blood pressure surge identification unit 154 identifies that the blood pressure waveform that is the blood pressure surge candidate is not a blood pressure surge. In another example, if there is at least one feature vector located inside of the boundary in the feature space, the blood pressure surge identification unit 154 may also identify that the blood pressure waveform that is the blood pressure surge candidate is a blood pressure surge.

Next, operations of the blood pressure data processing apparatus 10 will be described.

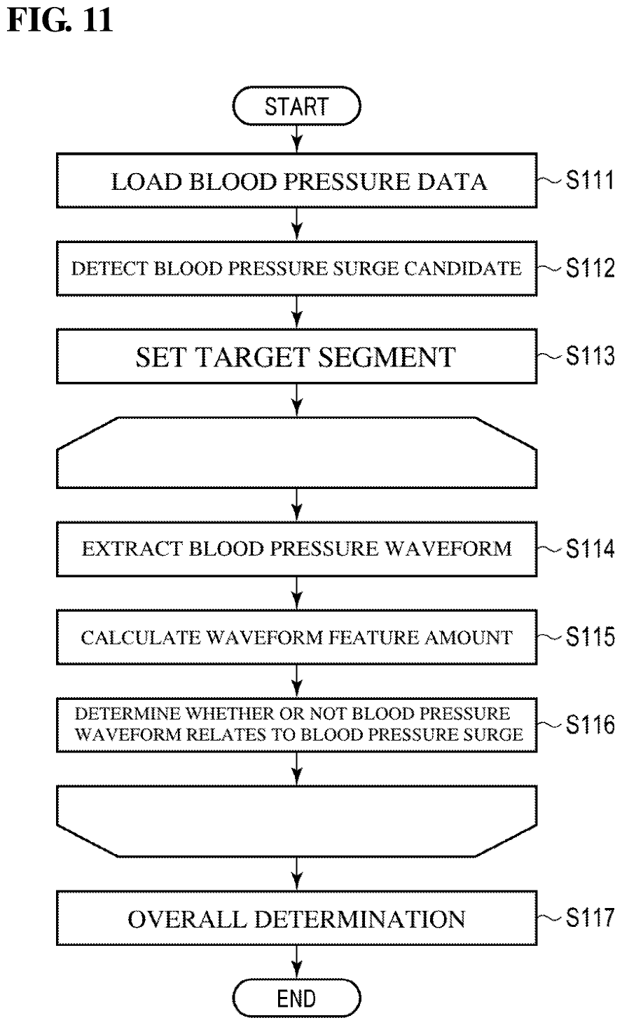

FIG. 11 shows an example of a procedure for identifying a blood pressure surge according to the first embodiment. In step S111 of FIG. 11, the blood pressure data is read out from the blood pressure data storage unit 12. In step S112, the blood pressure surge candidate detection unit 14 detects a blood pressure waveform that is a blood pressure surge candidate based on the blood pressure data. Here, in order to simplify the description, it is assumed that one blood pressure waveform that is a blood pressure surge candidate has been detected. If multiple blood pressure waveforms that are blood pressure surge candidates have been detected, the processing described hereinafter is executed for each blood pressure waveform.

In step S113, the blood pressure surge determination unit 15 sets a target segment for the blood pressure waveform that is the blood pressure surge candidate. In step S114, the blood pressure surge determination unit 15 extracts the blood pressure waveform of one or more heartbeats from the blood pressure waveform of the target segment. In step S115, the blood pressure surge determination unit 15 calculates the waveform feature amount based on the extracted blood pressure waveform of one or more heartbeats. Specifically, the blood pressure surge determination unit 15 calculates a waveform feature amount for each blood pressure waveform of one heartbeat isolated from the blood pressure waveform of one or more heartbeats, or for an average blood pressure waveform obtained by averaging the blood pressure waveforms of one heartbeat isolated from the blood pressure waveform of one or more heartbeats. In step S116, the blood pressure surge determination unit 15 identifies whether or not the respective blood pressure waveforms of one heartbeat or the average blood pressure waveform relates to a blood pressure surge based on the calculated waveform feature amount. For example, the blood pressure surge determination unit 15 identifies whether or not the respective blood pressure waveforms of one heartbeat or the average blood pressure waveform relates to the blood pressure surge based on a determination of whether the feature vectors representing the calculated waveform feature amounts are inside or outside of the boundary lines or planes set in the feature space.

If a blood pressure waveform of multiple heartbeats is included in the blood pressure waveform of the target segment extracted in step S113, the processing of steps S114 to S116 is performed on each blood pressure waveform of one heartbeat.

In step S117, the blood pressure surge determination unit 15 determines whether or not the blood pressure waveform that is the blood pressure surge candidate is a blood pressure surge based on the result of identification (step S116) that has been executed repeatedly. Specifically, if the number of instances of identifying that a blood pressure waveform is a blood pressure surge is greater than the number of instances of identifying that a blood pressure waveform is not a blood pressure surge, the blood pressure surge determination unit 15 determines that the blood pressure waveform that is the blood pressure surge candidate is a blood pressure surge, and if the number of instances of identifying that a blood pressure waveform is a blood pressure surge is less than the number of instances of identifying that a blood pressure waveform is not a blood pressure surge, the blood pressure surge determination unit 15 determines that the blood pressure waveform that is the blood pressure surge candidate is not a blood pressure surge.

As described above, the blood pressure data processing apparatus 10 extracts the blood pressure waveform of one or more heartbeats from the blood pressure waveform that is the blood pressure surge candidate, extracts the waveform feature amounts from the extracted blood pressure waveform of one or more heartbeats, and determines whether or not the blood pressure waveform that is the blood pressure surge candidate is a blood pressure surge based on the feature vectors indicating the calculated waveform feature amounts and the boundaries set in the feature space. Although there is no single definition for a blood pressure surge, it is possible to extract a typical blood pressure waveform that can be identified as a blood pressure surge from blood pressure data collected for the purpose of research, or the like. Boundaries are set in the feature space based on the typical blood pressure waveform extracted in this manner. Accordingly, it is possible to determine whether or not the blood pressure waveform that is the blood pressure surge candidate is a blood pressure surge using the waveform feature amount calculated based on the blood pressure waveform that is the blood pressure surge candidate. As a result, it is possible to determine whether a sudden blood pressure fluctuation included in the blood pressure data obtained through continuous blood pressure measurement is a blood pressure surge or noise, without any human intervention. Furthermore, the boundaries in the characteristic space can be determined based on a blood pressure surge waveform extracted from a blood pressure surge that is considered to be related to a specific disease. Accordingly, examination or treatment of a specific disease can be performed efficiently.

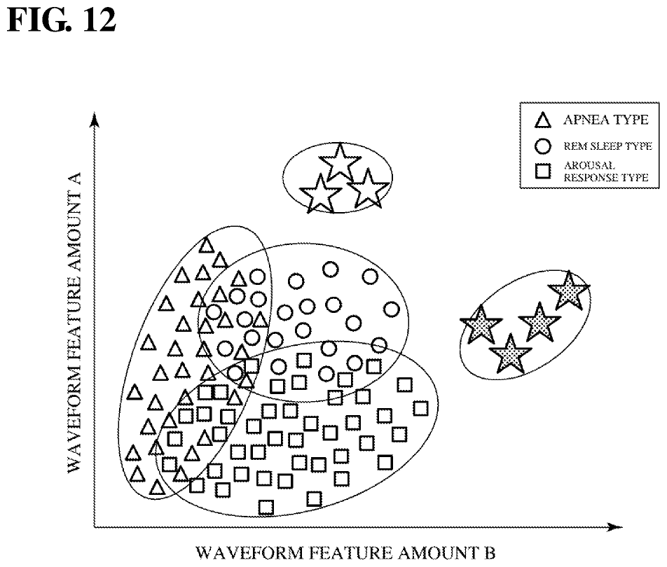

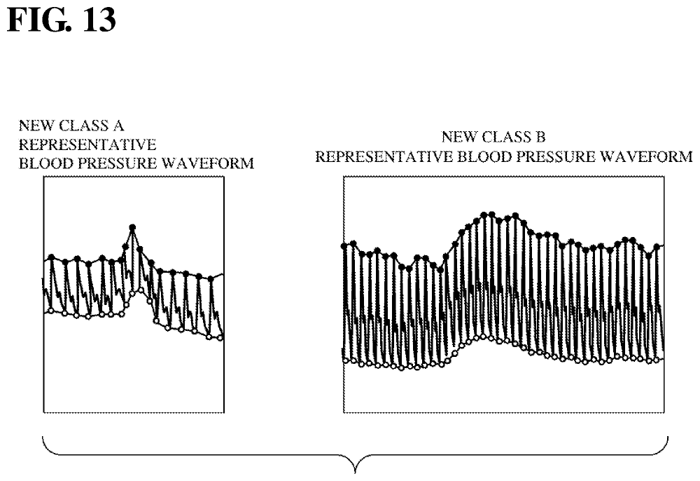

Information relating to a blood pressure waveform determined by the blood pressure surge identification unit 154 as not being a blood pressure surge can be fed back to the blood pressure surge identification data generation unit 155. The blood pressure surge identification data generation unit 155 can function as a clustering unit for clustering the blood pressure waveforms of one or more heartbeats extracted from the blood pressure waveform determined as not being a blood pressure surge in the feature space. In the example shown in FIG. 12, two classes have been newly generated. The newly-generated classes are each thought to be related to some kind of causal factor.

The information output unit 17 outputs a blood pressure waveform of one or more heartbeats located near (e.g., the closest to) the center of mass of each class as a representative blood pressure surge waveform. For example, as shown in FIG. 13, the blood pressure waveform including the representative blood pressure surge waveform is displayed for each new class. Furthermore, a message indicating that a new class has been detected may also be displayed.

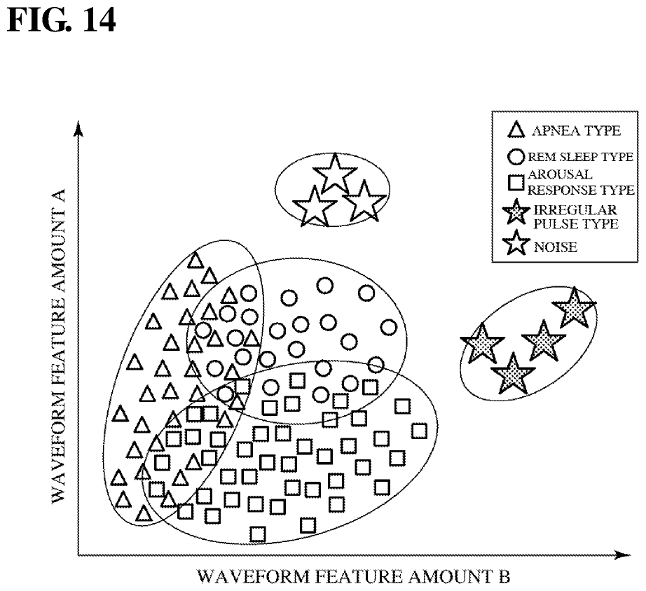

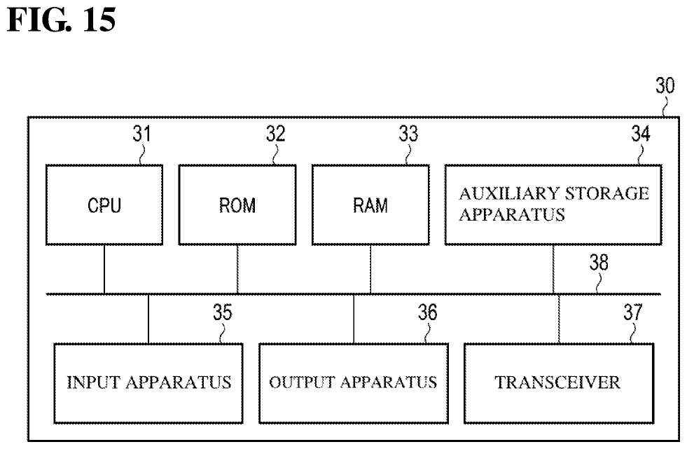

An expert analyzes the displayed blood pressure waveform and sets a new label for each class using a later-described input apparatus, for example. For example, as shown in FIG. 14, the label “irregular pulse type” is set for one class, and the label “noise” is set for the other. An irregular pulse increases the risk of onset of heart disease such as heart attack. Accordingly, the blood pressure surge identification data generation unit 155 provides the blood pressure surge identification unit 154 with blood pressure surge identification data including the boundaries for the three classes, namely apnea, REM sleep, and an arousal response, and the boundary for the irregular pulse class.

Thus, it is possible to generate a new class by performing clustering on blood pressure waveforms determined by the blood pressure surge identification unit 154 as not being blood pressure surges. An expert can discover a causal factor other than a causal factor that has already been defined by searching for blood pressure waveforms included in the new class.

An example of a hardware configuration of the blood pressure data processing apparatus 10 will be described with reference to FIG. 12.

The blood pressure data processing apparatus 10 includes a CPU 31, a ROM 32, a RAM 33, an auxiliary storage apparatus 34, an input apparatus 35, an output apparatus 36, and a transceiver 37, and these elements are connected to each other via a bus system 38. The above-described functions of the blood pressure data processing apparatus 10 can be realized by the CPU 31 reading out and executing a program stored in a computer-readable storage medium (the ROM 32 and/or the auxiliary storage apparatus 34). The RAM 33 is used as a work memory by the CPU 31. For example, the auxiliary storage apparatus 34 includes a hard disk drive (HDD) or a solid-state drive (SSD). The auxiliary storage apparatus 34 is used as the blood pressure data storage unit 12 (FIG. 1) and the blood pressure surge waveform storage unit 156 (FIG. 7). The input apparatus includes, for example, a keyboard, a mouse, and a microphone. The output apparatus includes, for example, a display apparatus such as a liquid crystal display apparatus and a speaker. The transceiver 37 performs transmission and reception of signals to and from another computer. For example, the transceiver 37 receives blood pressure data from the blood pressure measurement apparatus 20.

### Other Embodiments

In the above-described embodiment, the blood pressure surge identification data generation unit 155 and the blood pressure surge waveform storage unit 156 are provided in the blood pressure surge determination unit 15 of the blood pressure data processing apparatus 10. In another embodiment, the blood pressure surge identification data generation unit 155 and the blood pressure surge waveform storage unit 156 may also be provided in an apparatus different from the blood pressure data processing apparatus 10. In other words, the blood pressure surge identification data may also be generated in an external apparatus, and the blood pressure surge identification data may be provided to the blood pressure data processing apparatus 10.

Also, in the above-described embodiment, the blood pressure data processing apparatus 10 is provided separate from the blood pressure measurement apparatus 20. In another embodiment, a portion or all of the functions of the blood pressure data processing apparatus 10 may also be provided in the blood pressure measurement apparatus 20.

In short, the present invention is not limited to the above-described embodiment as-is, and can be realized with modifications to the constituent elements without departing from the gist in the implementation stage. Also, various aspects of the invention can be formed through suitable combinations of the multiple constituent elements disclosed in the above-described embodiment. For example, several constituent elements may also be removed from all of the constituent elements shown in the embodiment. Furthermore, the constituent elements of different embodiments may also be combined as appropriate.

A portion or the entirety of the above-described embodiment can be described as in the following supplementary notes as well, but there is no limitation to the following description.

Supplementary Note 1

A blood pressure data processing apparatus comprising:

a hardware processor; and

a memory coupled to the hardware processor,

wherein the hardware processor is configured to
acquire blood pressure data,
detect a blood pressure waveform that is a blood pressure surge candidate based on the blood pressure data,
extract a blood pressure waveform of one or more heartbeats from the blood pressure waveform that is the blood pressure surge candidate,
calculate a waveform feature amount for each blood pressure waveform of one heartbeat isolated from the blood pressure waveform of one or more heartbeats, or for an average blood pressure waveform obtained by averaging the blood pressure waveforms of one heartbeat isolated from the blood pressure waveform of one or more heartbeats, and
identify whether or not the blood pressure waveform that is the blood pressure surge candidate is a blood pressure surge based on the waveform characteristic amount.

Supplementary Note 2

A blood pressure data processing method comprising:

acquiring blood pressure data using at least one hardware processor;

detecting a blood pressure waveform that is a blood pressure surge candidate based on the blood pressure data using at least one hardware processor;

extracting a blood pressure waveform of one or more heartbeats from the blood pressure waveform that is the blood pressure surge candidate using at least one hardware processor;

calculating a waveform feature amount for each blood pressure waveform of one heartbeat isolated from the blood pressure waveform of one or more heartbeats, or for an average blood pressure waveform obtained by averaging the blood pressure waveforms of one heartbeat isolated from the blood pressure waveform of one or more heartbeats, using at least one hardware processor; and

identifying whether or not the blood pressure waveform that is the blood pressure surge candidate is a blood pressure surge based on the waveform feature amount using at least one hardware processor.

1. A blood pressure data processing apparatus comprising:
a blood pressure data acquisition unit configured to acquire blood pressure data;
a blood pressure surge candidate detection unit configured to detect a blood pressure waveform that includes a rising portion in which a blood pressure feature amount rises over a plurality of heartbeats, as a blood pressure surge candidate, based on the blood pressure data;
a blood pressure waveform extraction unit configured to extract a blood pressure waveform of one or more heartbeats from the rising portion of the blood pressure waveform that is the blood pressure surge candidate;
a waveform feature amount calculation unit configured to calculate a waveform feature amount for each blood pressure waveform of one heartbeat isolated from the blood pressure waveform of one or more heartbeats, or for an average blood pressure waveform obtained by averaging the blood pressure waveforms of one heartbeat isolated from the blood pressure waveform of one or more heartbeats; and
a blood pressure surge identification unit configured to identify whether or not the blood pressure waveform that is the blood pressure surge candidate is a blood pressure surge caused by a predetermined causal factor, based on the waveform feature amount.

2. The blood pressure data processing apparatus according to claim 1, wherein
the waveform feature amount includes a plurality of types of waveform feature amounts, and
the blood pressure surge identification unit identifies whether or not the blood pressure waveform that is the blood pressure surge candidate is the blood pressure surge based on the plurality of types of waveform feature amounts and a boundary set in a feature space.

3. The blood pressure data processing apparatus according to claim 1, wherein the blood pressure waveform of the blood pressure surge candidate further includes a falling portion that is subsequent to the rising portion.

4. The blood pressure data processing apparatus according to claim 1, wherein the waveform feature amount is based on at least one of a time interval from a time of a diastolic peak to a time of a systolic peak, a time interval from the time of the diastolic peak to a time of a dicrotic peak, a time width of the systolic peak, a total pulse time, an amplitude of the systolic peak, and an amplitude of the dicrotic peak.

5. The blood pressure data processing apparatus according to claim 4, wherein the waveform feature amount includes a waveform feature amount obtained based on a ratio between the time width of the systolic peak and the total pulse time.

6. The blood pressure data processing apparatus according to claim 4, wherein the waveform feature amount calculation unit performs pre-processing including a first derivative or a second derivative on the blood pressure waveform of one or more heartbeats, and specifies the diastolic peak, the systolic peak, and the dicrotic peak based on the waveform obtained through the pre-processing.

7. The blood pressure data processing apparatus according to claim 1, further comprising:
a display unit configured to display a blood pressure waveform identified by the blood pressure surge identification unit as not being the blood pressure surge; and
a reception unit configured to receive an instruction indicating whether or not the displayed blood pressure waveform is the blood pressure surge.

8. The blood pressure data processing apparatus according to claim 1, further comprising:
a clustering unit configured to generate a class by performing clustering on blood pressure waveforms of one or more heartbeats included in blood pressure waveform identified by the blood pressure surge identification unit as not being a the blood pressure surge; and
an output unit configured to output information including a blood pressure waveform of one or more heartbeats representing the class.

9. A blood pressure data processing method comprising:
acquiring blood pressure data;
detecting a blood pressure waveform that includes a rising portion in which a blood pressure feature amount rises over a plurality of heartbeats, as a blood pressure surge candidate, based on the blood pressure data;
extracting a blood pressure waveform of one or more heartbeats from the rising portion of the blood pressure waveform that is the blood pressure surge candidate;
calculating a waveform feature amount for each blood pressure waveform of one heartbeat isolated from the blood pressure waveform of one or more heartbeats, or for an average blood pressure waveform obtained by averaging the blood pressure waveforms of one heartbeat isolated from the blood pressure waveform of one or more heartbeats; and
identifying whether or not the blood pressure waveform that is the blood pressure surge candidate is a blood pressure surge caused by a predetermined causal factor, based on the waveform feature amount.

10. A program for causing a computer to function as the blood pressure data processing apparatus according to claim 1.
